(19) United States
(12) Patent Application Publication
(10) Pub. No.: US 2025/0155398 A1
(43) Pub. Date: May 15, 2025

(54) GAS SENSOR

(71) Applicant: NITERRA CO., LTD., Nagoya-shi, Aichi (JP)

(72) Inventors: Masahiro YAMASHITA, Nagoya-shi, Aichi (JP); Shoji KITANOYA, Nagoya-shi, Aichi (JP)

(73) Assignee: NITERRA CO., LTD., Nagoya-shi, Aichi (JP)

(21) Appl. No.: 18/726,317

(22) PCT Filed: Jan. 30, 2023
(86) PCT No.: PCT/JP2023/002841

§ 371 Date: Jan. 17, 2025

(30) Foreign Application Priority Data
Feb. 25, 2022 (JP) 2022-027496

(51) Int. Cl. G01N 27/18; G01N 27/04; G01N 33/00; G01R 35/02

(52) U.S. Cl. CPC G01N 27/18; G01N 27/045; G01R 35/02; G01N 33/005

(57) ABSTRACT

Gas sensor including first detection element 10 exposed to a reference atmosphere obtained by removing hydrogen gas from a target atmosphere, and second detection element 20 exposed to the target atmosphere. The first and second detection elements each having a resistance value changing in response to its temperature change connected in series, forming one side of bridge circuit 41. The sensor includes amplifying circuit 42 configured to output amplified voltage Vd2 based on an intermediate voltage between the first and second detection elements when a predetermined voltage is applied to the bridge circuit, non-amplifying circuit 43 configured to output the intermediate voltage as it is as non-amplified voltage Vd1, and control unit 30 to which the amplified and non-amplified voltages are inputted. The control unit calculates hydrogen gas concentration Dc (to-be-detected gas) based on the inputted amplified voltage, and determines failure of the sensor (e.g., based on the non-amplified voltage).

### TECHNICAL FIELD

The present disclosure relates to a gas sensor.

### BACKGROUND ART

As one example of a gas sensor, a hydrogen gas sensor described in Patent Document 1 below is known. This gas sensor includes a first gas detection element and a second gas detection element which are connected in series to form a bridge circuit and whose resistance values change in response to own temperature changes thereof, a current detection unit which detects a current flowing through these gas detection elements, and a calculation unit which calculates a hydrogen gas concentration from the potential between these gas detection elements. The first gas detection element is a reference element exposed to the atmosphere to be detected from which hydrogen gas has been removed, and the second gas detection element is a detection element exposed to the atmosphere to be detected as is. If the current detected by the current detection unit is equal to or higher than a threshold value, the calculation unit determines that the concentration of hydrogen gas in the atmosphere to be detected is high.

In the gas sensor as described above, for example, if one of the gas detection elements connected in series is damaged and short-circuited, the current flowing through the gas detection element may increase and the current detection value by the current detection unit may become equal to or higher than the threshold value. In the above gas sensor, it is difficult to distinguish between a state where the current detection value is equal to or higher than the threshold value due to damage of a gas detection element as described above and a state where the concentration of hydrogen gas in the atmosphere to be detected is actually very high. In the above gas sensor, the output of each gas detection element is amplified in order to increase the sensitivity of the gas sensor, and it is also difficult to determine a failure of such an amplifying circuit.

### RELATED ART DOCUMENT

Patent Document 1: Japanese Unexamined Patent Application Publication No. 2019-53028

### Problem to be Solved by the Invention

In view of the above situation, an object of this technology is to provide a gas sensor capable of failure diagnosis.

### DISCLOSURE OF THE PRESENT INVENTION

### Means for Solving the Problem

A gas sensor according to the present disclosure is a gas sensor for detecting a to-be-detected gas contained in a target atmosphere, the gas sensor comprising: a first detection element and a second detection element connected in series to form one side of a bridge circuit, the first detection element being exposed to a reference atmosphere obtained by removing the to-be-detected gas from the target atmosphere and having a resistance value changing in response to own temperature change thereof, the second detection element being connected in series to the first detection element, exposed to the target atmosphere, and having a resistance value changing in response to own temperature change thereof; an amplifying circuit configured to output an amplified voltage on the basis of an intermediate voltage between the first detection element and the second detection element when a predetermined voltage is applied to the bridge circuit; a non-amplifying circuit configured to output the intermediate voltage as it is as a non-amplified voltage; and a control unit to which the amplified voltage and the non-amplified voltage are inputted, wherein the control unit calculates a concentration of the to-be-detected gas on the basis of the inputted amplified voltage, and determines presence or absence of a failure of the gas sensor on the basis of at least the non-amplified voltage.

### Advantageous Effect of the Invention

According to the present disclosure, it is possible to provide a gas sensor capable of failure diagnosis.

### BRIEF DESCRIPTION OF THE DRAWINGS

FIG. 1 is a cross-sectional view schematically showing a schematic configuration of a gas sensor according to an embodiment.

FIG. 2 is a partially enlarged view of an area around detection elements in FIG. 1.

FIG. 3 is a plan view schematically showing a schematic configuration of the detection element.

FIG. 4 is an I-I cross-sectional view of FIG. 3.

FIG. 5 is a circuit diagram showing an example of an electrical configuration of the gas sensor and a display unit.

FIG. 6 is a flowchart showing an example of a process for detecting a failure of the detection element (Process Example 1).

FIG. 7 is a flowchart showing an example of a process for detecting a failure of an amplifying circuit (Process Example 2).

### BEST MODE FOR CARRYING OUT THE INVENTION

### Description of Embodiments of the Present Disclosure

First, embodiments of the present disclosure are listed.

<1> A gas sensor of the present disclosure is a gas sensor for detecting a to-be-detected gas contained in a target atmosphere, the gas sensor including: a first detection element and a second detection element connected in series to form one side of a bridge circuit, the first detection element being exposed to a reference atmosphere obtained by removing the to-be-detected gas from the target atmosphere and having a resistance value changing in response to own temperature change thereof, the second detection element being connected in series to the first detection element, exposed to the target atmosphere, and having a resistance value changing in response to own temperature change thereof; an amplifying circuit configured to output an amplified voltage on the basis of an intermediate voltage between the first detection element and the second detection element when a predetermined voltage is applied to the bridge circuit; a non-amplifying circuit configured to output the intermediate voltage as it is as a non-amplified voltage; and a control unit to which the amplified voltage and the non-amplified voltage are inputted, wherein the control unit calculates a concentration of the to-be-detected gas on the basis of the inputted amplified voltage, and determines presence or absence of a failure of the gas sensor on the basis of at least the non-amplified voltage.

With the configuration of <1> above, the control unit is capable of detecting the concentration of the to-be-detected gas with high sensitivity on the basis of the amplified voltage. In addition to the presence or absence of a failure in the detection element or in the amplifying circuit, the control unit can determine the location of the failure on the basis of the non-amplified voltage.

<2> In the gas sensor of <1> above, the non-amplifying circuit is configured to be able to output the non-amplified voltage within a predetermined non-amplified output range to the control unit, and if the inputted non-amplified voltage is a lower limit value or an upper limit value of the non-amplified output range, the control unit determines that a failure has occurred in at least either the first detection element or the second detection element.

With the configuration of <2> above, by referring to the non-amplified voltage, the control unit can determine the presence or absence of a failure of the detection element, which cannot be determined solely from the to-be-detected gas concentration calculated from the amplified voltage.

<3> In the holding device of <1> or <2> above, the control unit calculates an amplification factor on the basis of the inputted amplified voltage and the inputted non-amplified voltage, and determines that a failure has occurred in the amplifying circuit, if the amplification factor is outside an expected range.

With the configuration of <3> above, the control unit can determine the presence or absence of a failure of the amplifying circuit by calculating the amplification factor from the amplified voltage and the non-amplified voltage and comparing the amplification factor with the expected range.

### Details of Embodiments of the Present Disclosure

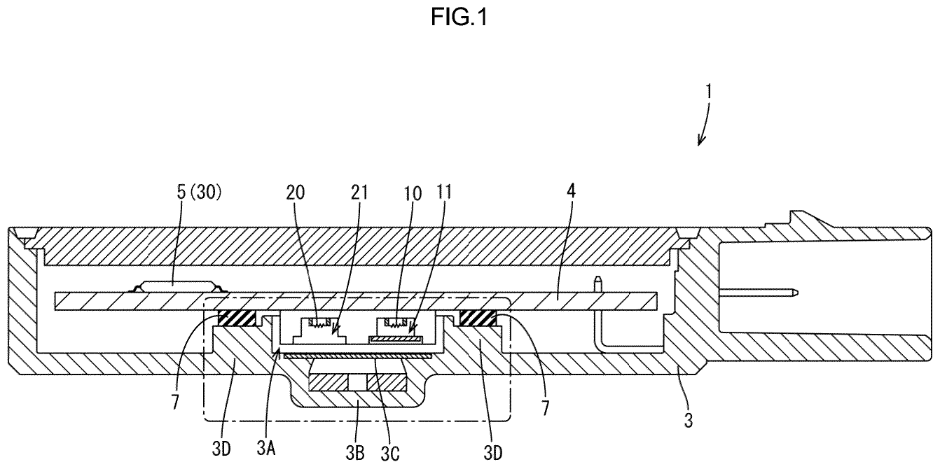

Specific examples of the gas sensor of the present disclosure will be described below with reference to the drawings. The present disclosure is not limited to these examples, but is indicated by the claims, and is intended to include all modifications within the meaning and scope equivalent to the claims. In the following description, the upper side of FIG. 1 is defined as upper side, and the lower side of FIG. 1 is defined as lower side, but this does not mean that the gas sensor is always installed in such an orientation. In each drawing, for a plurality of identical members, one member may be designated by a reference symbol and reference symbols for the other members may be omitted. In addition, the relative sizes and placement of components in each drawing are not necessarily accurate, and the scale or the like of some components has been changed for convenience of description.

### Details of Embodiments

Hereinafter, a configuration of a gas sensor 1 according to an embodiment will be described with reference to FIG. 1 to FIG. 5. The gas sensor 1 according to the present embodiment is designed to detect hydrogen gas contained in a target atmosphere as a to-be-detected gas, and can be installed, for example, inside a fuel cell vehicle and be used for, for example, the purpose of detecting hydrogen leakage.

### (Gas Sensor)

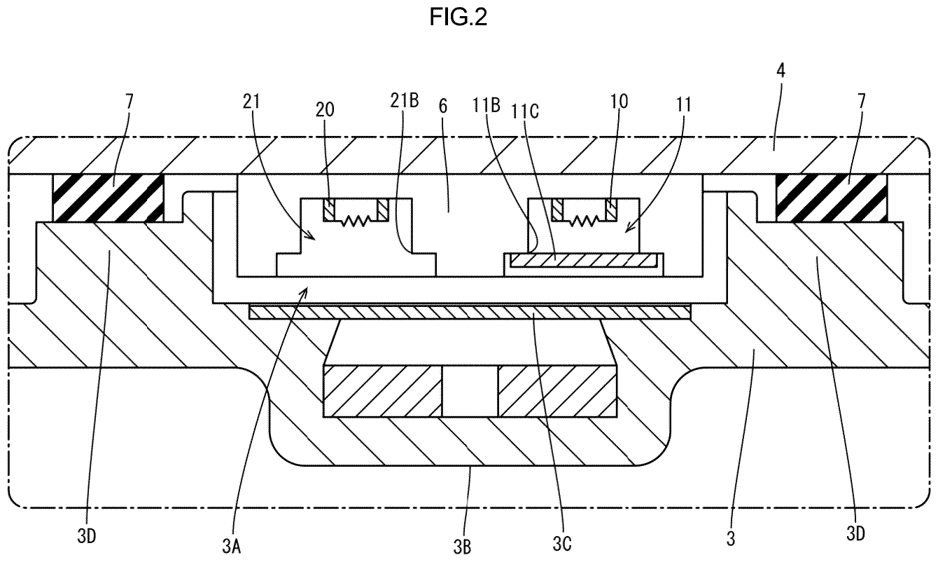

First, a schematic configuration of the gas sensor 1 will be described with reference to FIG. 1 and FIG. 2.

As shown in FIG. 1, etc., the gas sensor 1 includes a casing 3. A portion of the lower surface of the casing 3 protrudes downward, and a gas introduction port 3B is formed in this protruding portion so as to connect an internal space 3A formed inside the casing 3 and the outside of the casing 3. A filter 3C is placed at the gas introduction port 3B. The filter 3C is a water repellent filter that allows the target atmosphere to pass therethrough and does not allow liquid water to pass therethrough (i.e., removes water droplets contained in the target atmosphere). The filter 3C is attached to the inner surface of the casing 3 so as to cover the gas introduction port 3B. This configuration allows the target atmosphere containing hydrogen gas (to-be-detected gas) to flow from the gas introduction port 3B to the internal space 3A, and also inhibits entry of water droplets and other foreign matter. In addition, an inner frame 3D is provided around the gas introduction port 3B so as to protrude toward the inner side of the casing 3.

As shown in FIG. 1, etc., a circuit board 4 on which a microcomputer 5 is mounted is housed inside the casing 3 in an orientation in which a board surface thereof faces the up-down direction. The lower board surface of the circuit board 4 is fixed to the inner frame 3D via a seal member 7, and the microcomputer 5 is mounted on the upper board surface of the circuit board 4.

As shown in FIG. 2 in an enlarged manner, in the casing 3, the internal space 3A defined by the filter 3C placed at the gas introduction port 3B, the inner frame 3D, the seal member 7, and the circuit board 4 is provided with a first housing portion 11 in which a first detection element 10 is housed and a second housing portion 21 in which a second detection element 20 is housed.

As shown in FIG. 2, a base 6 is installed on the lower board surface of the circuit board 4, and the first housing portion 11 and the second housing portion 21 are formed by two recesses provided on the lower surface of the base 6, respectively. As the base 6, for example, one made of an insulating ceramic such as alumina, aluminum nitride, and zirconia can be suitably used. The first housing portion 11 and the second housing portion 21 are adjacent to both sides of one isolation wall formed by the base 6, and are separated from each other so as not to communicate with each other. Since the first housing portion 11 and the second housing portion 21 are provided close to each other as described above, the temperature difference between the interior of the first housing portion 11 and the interior of the second housing portion 21 can be reduced, thereby reducing the output fluctuation of the gas sensor 1 with respect to temperature change and reducing errors in the sensor output.

As shown in FIG. 2, the first housing portion 11 and the second housing portion 21 are both open downward, that is, on the filter 3C side. Hereinafter, the opening of the first housing portion 11 is referred to as first opening 11B, and the opening of the second housing portion 21 is referred to as second opening 21B. The first opening 11B and the second opening 21B each connect the first housing portion 11 or the second housing portion 21 and the internal space 3A of the casing 3 which is the outside of the respective housing portions 11 and 21. The first housing portion 11 has no opening except for the first opening 11B, and the second housing portion 21 has no opening except for the second opening 21B.

As shown in FIG. 2, a membrane body 11C is installed at the first opening 11B. The membrane body 11C is made of a solid polymer electrolyte having a property of being permeable to water vapor and less permeable to hydrogen gas than water vapor (i.e., a property of inhibiting permeation of hydrogen gas). As such a membrane body 11C, a fluorine resin-based ion exchange membrane can be suitably used. Specific examples of such a membrane include Nafion (registered trademark), Flemion (registered trademark), Aciplex (registered trademark), etc. As the membrane body 11C, a hollow fiber membrane capable of separating hydrogen gas and water vapor may also be used.

As shown in FIG. 2, the membrane body 11C is adhered at a periphery thereof to a step portion, of the base 6, which is formed at the first opening 11B, by an insulating adhesive so as to cover and seal the entire first opening 11B. Accordingly, inflow of hydrogen gas into the interior of the first housing portion 11 is restricted, and a reference atmosphere obtained by removing hydrogen gas from the target gas is supplied to the interior of the first housing portion 11. As a result, the first detection element 10 which is housed inside the first housing portion 11 functions as a reference element exposed to the reference atmosphere.

Meanwhile, the second opening 21B is opened toward the internal space 3A. Therefore, the target atmosphere containing hydrogen gas is supplied from the internal space 3A of the casing 3 to the interior of the second housing portion 21 through the second opening 21B without flowing through a membrane body or the like. Accordingly, the second detection element 20 which is housed inside the second housing portion 21 functions as a measurement element exposed to the target atmosphere containing hydrogen gas. Since the membrane body 11C allows water vapor to permeate therethrough as described above, the humidity conditions inside the first housing portion 11 and the second housing portion 21 are the same, so that the influence of humidity on gas concentration measurement is reduced.

### (Detection Elements)

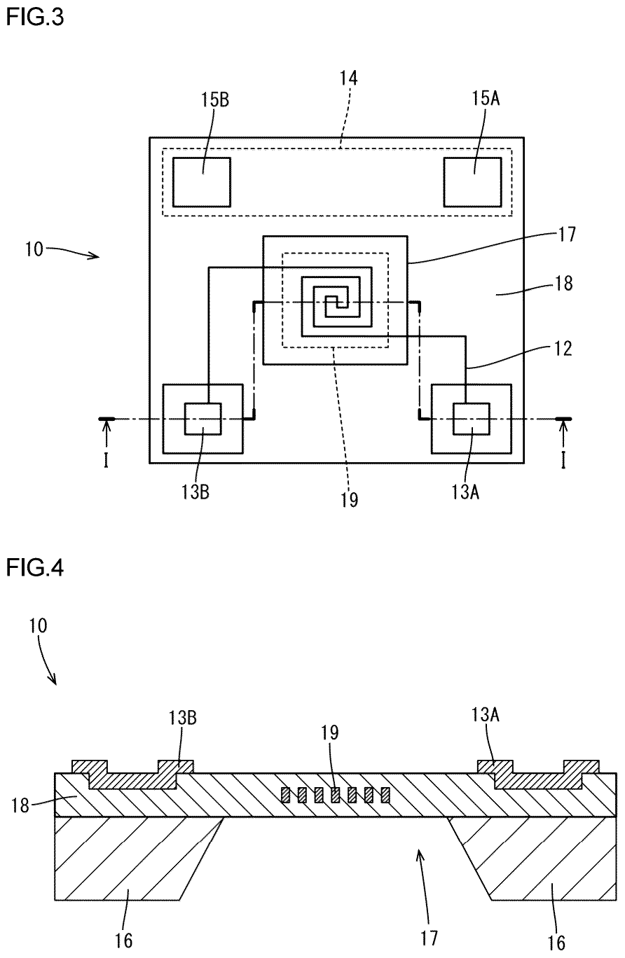

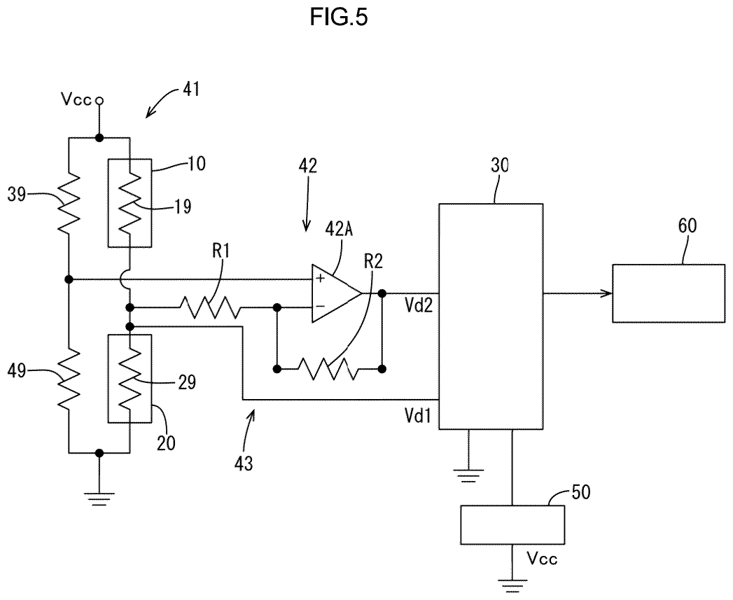

Next, the first detection element 10 and the second detection element 20 which are main parts of the gas sensor 1 will be described with reference to FIG. 3 and FIG. 4. The first detection element 10 and the second detection element 20 are each a thermal conduction type gas detection element and each have a heat generating resistor whose resistance value changes in response to own temperature change thereof. The use of a thermal conduction type detection element enables accurate determination of even a high-concentration hydrogen gas environment where there is a risk of oxygen shortage.

The first detection element 10 and the second detection element 20 have the same configuration. Thus, the first detection element 10 is mainly described below, and the detailed description of the second detection element 20 is omitted.

As shown in FIG. 3 and FIG. 4, the first detection element 10 includes a first heat generating resistor 19, an insulating layer 18, a wire 12, a pair of first electrode pads 13A and 13B, a temperature measuring resistor 14, a pair of second electrode pads 15A and 15B, and a substrate 16.

As shown in FIG. 3, the first heat generating resistor 19 is a conductor patterned in a spiral shape, and is electrically connected to the first electrode pads 13A and 13B via the wire 12. As shown in FIG. 4, the first heat generating resistor 19 is embedded in a center portion of the insulating layer 18. The first heat generating resistor 19 is a member whose resistance value changes in response to own temperature change thereof, and is made of a conductive material having a large temperature resistance coefficient. As the material of the first heat generating resistor 19, for example, platinum (Pt) can be used. The wire 12 can also be formed using the same material as the first heat generating resistor 19.

Hereinafter, a conductor placed in the second detection element 20 in the same manner as the first heat generating resistor 19 in the first detection element 10 is sometimes referred to as second heat generating resistor 29. In the present embodiment, as each of the first heat generating resistor 19 and the second heat generating resistor 29, one having the same resistance value is used.

As shown in FIG. 4, the first electrode pads 13A and 13B are formed on a surface of the insulating layer 18, and one of the first electrode pads 13A and 13B is connected to a ground. The first electrode pads 13A and 13B can be formed using the same material as the first heat generating resistor 19.

As shown in FIG. 4, the substrate 16 made of silicon is stacked on the surface of the insulating layer 18 on the side opposite to the first electrode pads 13A and 13B, except for a region where the first heat generating resistor 19 is placed. A region where the substrate 16 is not placed is a recess 17 where the insulating layer 18 is exposed, and forms a diaphragm structure. The insulating layer 18 may be formed of a single material, or may be composed of a plurality of layers for which different materials are used. Examples of the insulating material forming the insulating layer 18 include silicon oxide (SiO₂), silicon nitride (Si₃N₄), etc.

As shown in FIG. 3, the temperature measuring resistor 14 is embedded on the outer edge side of the insulating layer 18 with respect to the first heat generating resistor 19 in a plan view, and is electrically connected to the second electrode pads 15A and 15B. Specifically, the temperature measuring resistor 14 is placed near one side of the insulating layer 18. In addition, one of the second electrode pads 15A and 15B is connected to the ground. The temperature measuring resistor 14 is made of a conductive material whose resistance value changes in proportion to temperature. As the material of the temperature measuring resistor 14, for example, platinum (Pt) which is the same as in the first heat generating resistor 19 can be used. The material of the second electrode pads 15A and 15B can also be the same as the first heat generating resistor 19.

### (Circuit and Control Unit)

Next, an electrical configuration of the gas sensor 1 will be described with reference to FIG. 5. A portion of a circuit shown in FIG. 5 excluding a display unit 60 is formed in the circuit board 4, etc., which is placed inside the casing 3 as already described. This circuit includes a bridge circuit 41, an amplifying circuit 42 which amplifies an intermediate voltage (potential difference) detected by the bridge circuit 41, and a non-amplifying circuit 43 which outputs the intermediate voltage detected by the bridge circuit 41 as it is.

The bridge circuit 41 is electrically connected to each of the first electrode pads 13A and 13B and the second electrode pads 15A and 15B of the first detection element 10 and the second detection element 20. The first detection element 10 and the second detection element 20 are connected in series and form one side of the bridge circuit 41. Two resistors forming another side in the bridge circuit 41 are fixed resistors 39 and 49 having constant resistance values, respectively. Thus, by measuring a divided voltage at the bridge circuit 41, the change in the intermediate voltage between the first detection element 10 and the second detection element 20 can be read.

The amplifying circuit 42 is a well-known inverting amplifier circuit composed of an operational amplifier 42A, a fixed resistor R1 connected to the inverting input terminal of the operational amplifier 42A, and a fixed resistor R2 connected in parallel between the inverting input terminal and the output terminal of the operational amplifier 42A. An amplification factor A of such an amplifying circuit 42 is theoretically given by R2/R1 (A=R2/R1). The amplifying circuit 42 amplifies the intermediate voltage between the first detection element 10 and the second detection element 20 with the voltage between the fixed resistors 39 and 49 as a reference voltage, and further outputs a voltage offset by the reference voltage, as an amplified voltage Vd2 toward a control unit 30.

The non-amplifying circuit 43 does not include an operational amplifier, etc., and outputs the intermediate voltage between the first detection element 10 and the second detection element 20 as it is, as a non-amplified voltage Vd1 toward the control unit 30.

The microcomputer 5 which is mounted on the circuit board 4 as already described includes the control unit 30 which executes various processes. The control unit 30 performs a process related to calculation of a hydrogen gas concentration D and failure determination of the gas sensor 1 on the basis of an inputted voltage Vd, and outputs the calculated hydrogen gas concentration D and a determination signal to a vehicle-side ECU. Usually, the control unit 30 is designed to be able to calculate the hydrogen gas concentration D within a predetermined range.

The display unit 60 shown in FIG. 5 is provided in the vehicle-side ECU, and the hydrogen gas concentration D and a determination result are displayed on the display unit 60 on the basis of output signals from the vehicle-side ECU. The display unit 60 includes, for example, a panel, etc. The hydrogen gas concentration D may be displayed on the display unit 60 as a graph of transition over time. The display unit 60 may be configured to emit light or emit a warning sound, etc., when necessary, in addition to performing display by means of images. The hydrogen gas concentration D and the determination result may be outputted from the control unit 30 directly to the display unit 60 or the like not via the vehicle-side ECU, and may be displayed on the display unit 60 or the like.

A current is supplied from a DC power supply 50 to the circuit board 4 and the microcomputer 5, and a constant application voltage Vcc is applied to the first detection element 10 and the second detection element 20 which are connected in series in the bridge circuit 41.

The control unit 30 calculates the hydrogen gas concentration D in the target atmosphere on the basis of the amplified voltage Vd2 inputted from the amplifying circuit 42. In the present specification, the hydrogen gas concentration D calculated on the basis of the amplified voltage Vd2 is sometimes referred to as hydrogen gas concentration Dc.

The first detection element 10 which is exposed to the reference atmosphere obtained by removing hydrogen gas from the target atmosphere and the second detection element 20 which is exposed to the target atmosphere containing hydrogen gas each have the first heat generating resistor 19 or second heat generating resistor 29 whose resistance value changes in response to own temperature change thereof. Thus, when the application voltage Vcc is constant, the intermediate voltage between the first detection element 10 and the second detection element 20 varies in accordance with the hydrogen gas concentration D in the target atmosphere.

The control unit 30 according to the present embodiment is configured to be able to detect the hydrogen gas concentration D with high accuracy by acquiring the amplified voltage Vd2, which is obtained by amplifying the intermediate voltage by the amplifying circuit 42, and calculating the hydrogen gas concentration Dc on the basis of the amplified voltage Vd2. When the hydrogen gas concentration D becomes high, the control unit 30, which is designed to be able to calculate the hydrogen gas concentration D within a predetermined concentration range, calculates the hydrogen gas concentration Dc as an upper limit value Dcmax if the amplified voltage Vd2 outputted from the amplifying circuit 42 increases to be equal to or higher than a threshold value. However, even when a failure occurs in the first detection element 10 or the second detection element 20, the amplified voltage Vd2 inputted to the control unit 30 abnormally increases to be equal to or higher than the threshold value, so that the hydrogen gas concentration Dc is maintained at the upper limit value Dcmax. Alternatively, even when a failure occurs in the amplifying circuit 42, the amplified voltage Vd2 inputted to the control unit 30 may increase and the hydrogen gas concentration Dc may be maintained at the upper limit value Dcmax. Therefore, if whether or not the hydrogen gas concentration D is high is determined on the basis of only the hydrogen gas concentration Dc, there is a possibility that a false warning is issued when the hydrogen gas concentration Dc becomes the upper limit value Dcmax due to a failure of the detection element 10 or 20 or the amplifying circuit 42 even though the actual hydrogen gas concentration D is not high.

Similarly, if whether or not the hydrogen gas concentration D is low is determined on the basis of only the hydrogen gas concentration Dc, there is a possibility that it is determined that the hydrogen gas concentration D is low, when the hydrogen gas concentration Dc becomes a lower limit value Dcmin due to a failure of the detection element 10 or 20 or the amplifying circuit 42 even though the actual hydrogen gas concentration D is not low.

Therefore, the control unit 30 according to the present embodiment is configured to be able to determine whether or not a failure has occurred in the first detection element 10 or the second detection element 20 or in the amplifying circuit 42, on the basis of the non-amplified voltage Vd1 inputted from the non-amplifying circuit 43. For example, the control unit 30 can be set to execute a process for performing failure determination when the hydrogen gas concentration Dc which is calculated on the basis of the amplified voltage Vd2 as described above is the upper limit value Dcmax or the lower limit value Dcmin.

### Process Example 1: Failure Determination of Detection Element

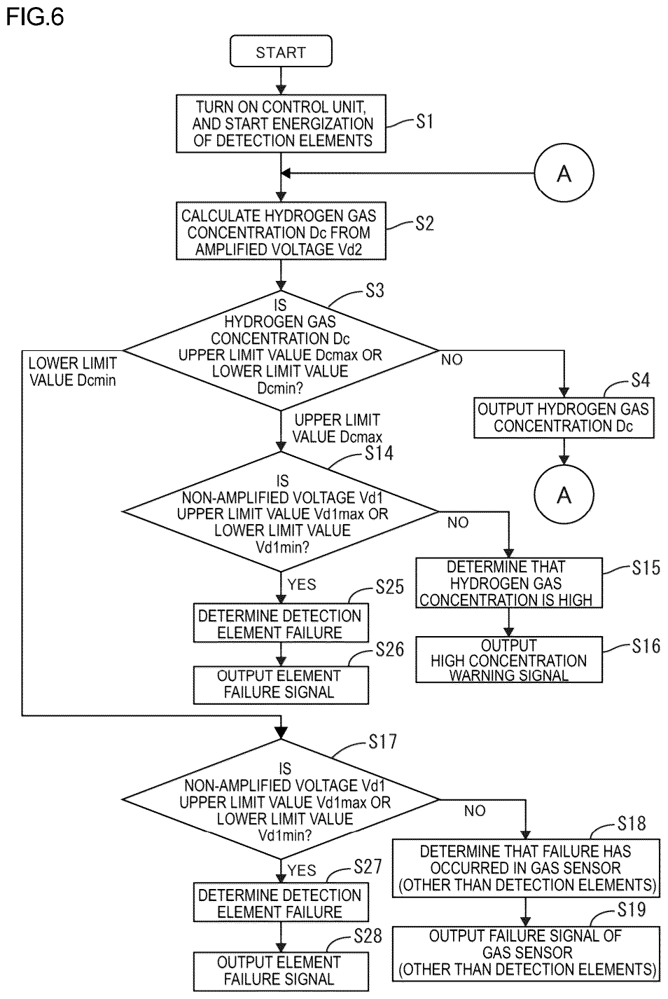

Hereinafter, one example of a process executed by the control unit 30 set to perform failure determination of the detection elements 10 and 20 when the hydrogen gas concentration Dc is the upper limit value Dcmax or the lower limit value Dcmin, will be described with reference to FIG. 6.

When power is supplied to the gas sensor 1, the control unit 30 is activated (step S1). The first detection element 10 and the second detection element 20 also start to be energized (step S1) and the constant application voltage Vcc is applied thereto.

When the intermediate voltage between the first detection element 10 and the second detection element 20 is amplified by the amplifying circuit 42 and the amplified voltage Vd2 is inputted to the control unit 30, the control unit 30 calculates the hydrogen gas concentration Dc on the basis of the inputted amplified voltage Vd2 (step S2). The control unit 30 may be equipped with a timer and configured to execute the calculation of the hydrogen gas concentration Dc each time a certain time elapses, or may be configured to execute the calculation of the hydrogen gas concentration Dc at any time.

In the gas sensor 1 according to the present embodiment, the amplified voltage Vd2 is represented by the following equation (1) using the amplification factor A, the non-amplified voltage (intermediate voltage) Vd1, and a reference voltage Vd0 which is an intermediate voltage between the fixed resistor 39 and the fixed resistor 49.

$$V d 2 = A ( V d 0 - V d 1 ) + V d 0 \qquad ( 1 )$$

In the present embodiment, the control unit 30 is configured to be able to calculate the hydrogen gas concentration Dc within a predetermined range, specifically within a range of the upper limit value Dcmax or lower. When the amplified voltage Vd2 based on which the hydrogen gas concentration Dc exceeds the upper limit value Dcmax is outputted from the amplifying circuit 42, that is, when the amplified voltage Vd2 is equal to or higher than a predetermined threshold value, the control unit 30 calculates the hydrogen gas concentration Dc as the upper limit value Dcmax. The threshold value for the amplified voltage Vd2 is a value set in advance in consideration of the environment in which the gas sensor 1 is installed (expected concentration range of the hydrogen gas concentration D), predicted measurement errors, the processing capability of the control unit 30, etc.

Next, the control unit 30 determines whether or not the hydrogen gas concentration Dc calculated as described above is the upper limit value Dcmax or the lower limit value Dcmin (step S3).

If the hydrogen gas concentration Dc is neither the upper limit value Dcmax nor the lower limit value Dcmin in step S3 (NO in step S3), the control unit 30 outputs the calculated hydrogen gas concentration Dc (step S4) and returns to step S2 to repeat the process.

If the calculated hydrogen gas concentration Dc is the upper limit value Dcmax in step S3 (the upper limit value Dcmax in step S3), the control unit 30 acquires the non-amplified voltage Vd1 from the non-amplifying circuit 43, and determines whether a failure has occurred in the detection element 10 or 20, on the basis of the non-amplified voltage Vd1.

Specifically, the control unit 30 compares the acquired non-amplified voltage Vd1 with an upper limit value Vd1max and a lower limit value Vd1min of a non-amplified output range that the non-amplified voltage Vd1 can take (step S14). In the bridge circuit 41, for example, if the detection element connected on the downstream side is short-circuited or the detection element connected on the upstream side is opened, the non-amplified voltage Vd1 becomes the lower limit value Vd1min. If the detection element connected on the upstream side is short-circuited or the detection element connected on the downstream side is opened, the non-amplified voltage Vd1 becomes the upper limit value Vd1max.

If the non-amplified voltage Vd1 is not the upper limit value Vd1max (Vd1≠Vd1max) and the non-amplified voltage Vd1 is not the lower limit value Vd1min (Vd1≠Vd1min) in step S14 (NO in step S14), the control unit 30 determines that no failure has occurred in the detection elements 10 and 20 and the hydrogen gas concentration D is very high (step S15), and outputs a high concentration warning signal (step S16). For example, when the high concentration warning signal is inputted to the vehicle-side ECU, a warning indication can be displayed or a warning sound can be emitted to alert a user.

If the non-amplified voltage Vd1 is the upper limit value Vd1max (Vd1=Vd1max) or the non-amplified voltage Vd1 is the lower limit value Vd1min (Vd1=Vd1min) in step S14 (YES in step S14), the control unit 30 determines that a failure has occurred in any of the detection elements 10 and 20 (step S25), and outputs an element failure signal (step S26). For example, when the element failure signal is inputted to the vehicle-side ECU, a failure indication of the first detection element 10 or the second detection element 20 can be displayed to urge the user to take action such as inspection or component replacement.

If the calculated hydrogen gas concentration Dc is the lower limit value Dcmin in step S3 (the lower limit value Dcmin in step S3), the control unit 30 acquires the non-amplified voltage Vd1 from the non-amplifying circuit 43, and determines whether a failure has occurred in the detection element 10 or 20, on the basis of the non-amplified voltage Vd1.

If the non-amplified voltage Vd1 is not the upper limit value Vd1max (Vd1≠Vd1max) and the non-amplified voltage Vd1 is not the lower limit value Vd1min (Vd1≠Vd1min) in step S17 (NO in step S17), the control unit 30 determines that a failure has occurred in the gas sensor 1 (other than the detection elements 10 and 20) (step S18), and outputs a failure signal of the gas sensor 1 (other than the detection elements 10 and 20) (step S19). For example, when the failure signal of the gas sensor 1 (other than the detection elements 10 and 20) is inputted to the vehicle-side ECU, a failure indication (other than the detection elements 10 and 20) can be displayed to urge the user to take action such as inspection or component replacement.

If the non-amplified voltage Vd1 is the upper limit value Vd1max (Vd1=Vd1max) or the non-amplified voltage Vd1 is the lower limit value Vd1min (Vd1=Vd1min) in step S17 (YES in step S17), the control unit 30 determines that a failure has occurred in any of the detection elements 10 and 20 (step S27), and outputs an element failure signal (step S28). For example, when the element failure signal is inputted to the vehicle-side ECU, a failure indication of the first detection element 10 or the second detection element 20 can be displayed to urge the user to take action such as inspection or component replacement.

### Specific Example of Process Example 1

As a specific example, the above process of the control unit 30 in the gas sensor 1 designed to be able to calculate the hydrogen gas concentration D within a range of 0 to 4% with the application voltage Vcc=5.0 V, the reference voltage Vd0=2.5 V, and the amplification factor A=20, is discussed. In such a gas sensor 1, Dcmax=4%, Dcmin=−0.8%, Vd1min=0 V, Vd1max=5.0 V, and the threshold value for the amplified voltage Vd2 is set to 3.75 V.

In such a gas sensor 1, for example, if the intermediate voltage is 2.0 V, the amplified voltage Vd2 outputted from the amplifying circuit 42 is Vd2=12.5 V from the aforementioned equation (1), and 5 V which is the upper limit value of the output of the operational amplifier 42A is outputted. If the amplified voltage Vd2 is equal to or higher than 3.75 V which is the threshold value, the control unit 30 calculates the hydrogen gas concentration Dc to be 4%, which is the upper limit value Dcmax, in step S2. Since Dc=Dcmax in step S3, failure determination of the detection elements 10 and 20 is performed in step S14. The non-amplified voltage Vd1 is 2.0 V, Vd1≠Vd1min, and Vd1≠Vd1max, so that NO is determined in step S14. Therefore, the control unit 30 determines in step S15 that no failure has occurred in the detection elements 10 and 20 and the hydrogen gas concentration Dc is very high, and outputs a high concentration warning signal in step S16.

In the gas sensor 1 as described above, for example, if the second detection element 20 which is connected on the downstream side is short-circuited, the intermediate voltage becomes 0 V. Referring to the aforementioned equation (1), the amplified voltage Vd2 outputted from the amplifying circuit 42 is Vd2=52.5 V, and Vd2>3.75 V. Thus, in step S2, the hydrogen gas concentration Dc is calculated to be 4% which is the upper limit value Dcmax. Since Dc=Dcmax in step S3, failure determination of the detection elements 10 and 20 is performed in step S14. The non-amplified voltage Vd1 is 0 V, and Vd1=Vd1min in step S14. Thus, the control unit 30 determines in step S25 that a failure has occurred in the detection element 10 or 20, and outputs a detection element failure signal in step S26.

In the gas sensor 1 as described above, for example, if the second detection element 20 which is connected on the downstream side is opened, the intermediate voltage is equal to the application voltage Vcc and becomes 5 V. Referring to the aforementioned equation (1), the amplified voltage Vd2 outputted from the amplifying circuit 42 is Vd2=−47.5 V, and 0 V which is the lower limit value of the output of the operational amplifier 42A is outputted. Since Vd2=0 V, the hydrogen gas concentration Dc is calculated to be −0.8%, which is the lower limit value Dcmin, in step S2. Since Dc=Dcmin in step S3, failure determination of the detection elements is performed in step S17. The non-amplified voltage Vd1 is 5 V, and Vd1=Vd1max, so that YES is determined in step S17. Therefore, the control unit 30 determines in step S27 that a failure has occurred in the detection element 10 or 20, and outputs an element failure signal in step S28.

### Process Example 2: Failure Determination of Amplifying Circuit

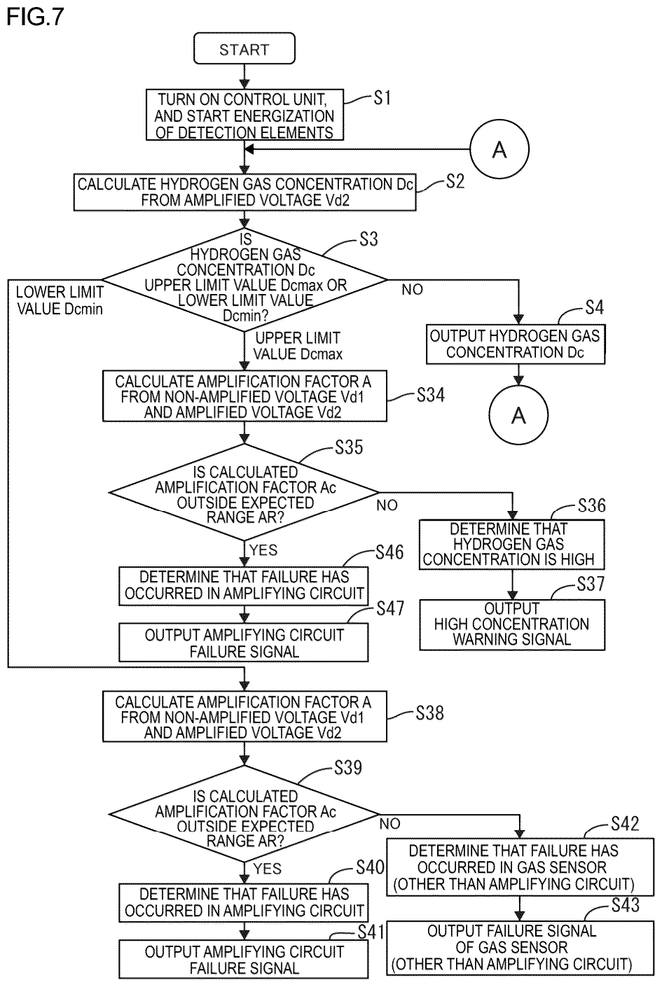

Next, one example of a process executed by the control unit 30 set to perform failure determination of the amplifying circuit 42 when the hydrogen gas concentration Dc is the upper limit value Dcmax or the lower limit value Dcmin, will be described with reference to FIG. 7. In this process example, processes from step S1 to step S4 are the same as in Process Example 1, and thus the same symbols are used and the description thereof is omitted.

If the hydrogen gas concentration Dc is the upper limit value Dcmax in step S3, the control unit 30 acquires the non-amplified voltage Vd1 from the non-amplifying circuit 43, and determines whether a failure has occurred in the amplifying circuit 42, on the basis of the non-amplified voltage Vd1 and the amplified voltage Vd2.

Specifically, the control unit 30 calculates the amplification factor A of the amplifying circuit 42 on the basis of the non-amplified voltage Vd1 and the amplified voltage Vd2 (step S34). In the present specification, the amplification factor A calculated from the non-amplified voltage Vd1 and the amplified voltage Vd2 is sometimes referred to as amplification factor Ac (Ac=(Vd2−2.5)/(2.5−Vd1)). Then, the control unit 30 compares the calculated amplification factor Ac with a preset expected range AR of the amplification factor A (step S35). The expected range AR of the amplification factor A is set in consideration of sensitivity errors in the first detection element 10 and the second detection element 20, A/D conversion errors in the control unit 30 and a circuit that reads and processes values measured by the first detection element 10 and the second detection element 20, etc. That is, the expected range AR is set in advance with a certain degree of width relative to the theoretical amplification factor A, taking into consideration variations in the values of the amplified voltage Vd2 and the non-amplified voltage Vd1 inputted to the control unit 30, noise, etc., and the expected range AR is stored in the control unit 30. When a failure occurs in the amplifying circuit 42, the amplification factor Ac is predicted to approach 1 or approach infinity. Therefore, it is preferable to set the expected range AR such that failure determination of the amplifying circuit 42 can be performed.

If, in step S35, the amplification factor Ac is within the expected range AR, that is, the amplification factor Ac is equal to or higher than a lower limit value Acmin of the expected range AR and equal to or lower than an upper limit value Acmax of the expected range AR (Acmin≤Ac≤Acmax) (NO in step S35), the control unit 30 determines that no failure has occurred in the amplifying circuit 42 and the hydrogen gas concentration D is very high (step S36), and outputs a high concentration warning signal (step S37).

If, in step S35, the amplification factor Ac is outside the expected range AR, that is, the amplification factor Ac is lower than the lower limit value Acmin of the expected range AR (Ac<Acmin), or the amplification factor Ac is higher than the upper limit value Acmax of the expected range AR (Ac>Acmax) (YES in step S35), the control unit 30 determines that a failure has occurred in the amplifying circuit 42 (step S46), and outputs an amplifying circuit failure signal (step S47).

If the hydrogen gas concentration Dc is the lower limit value Dcmin in step S3, the control unit 30 acquires the non-amplified voltage Vd1 from the non-amplifying circuit 43, and determines whether a failure has occurred in the amplifying circuit 42, on the basis of the non-amplified voltage Vd1 and the amplified voltage Vd2.

Specifically, the control unit 30 calculates the amplification factor A of the amplifying circuit 42 on the basis of the non-amplified voltage Vd1 and the amplified voltage Vd2 (step S38). In the present specification, the amplification factor A calculated from the non-amplified voltage Vd1 and the amplified voltage Vd2 is sometimes referred to as amplification factor Ac (Ac=(Vd2−2.5)/(2.5−Vd1)). Then, the control unit 30 compares the calculated amplification factor Ac with the preset expected range AR of the amplification factor A (step S39).

If, in step S39, the amplification factor Ac is within the expected range AR, that is, the amplification factor Ac is equal to or higher than the lower limit value Acmin of the expected range AR and equal to or lower than the upper limit value Acmax of the expected range AR (Acmin≤Ac≤Acmax) (NO in step S39), the control unit 30 determines that no failure has occurred in the amplifying circuit 42 but a failure has occurred in the gas sensor 1 (other than the amplifying circuit 42) (step S42), and outputs a failure signal of the gas sensor 1 (other than the amplifying circuit 42) (step S43). For example, when the failure signal of the gas sensor 1 (other than the amplifying circuit 42) is inputted to the vehicle-side ECU, a failure indication (other than the amplifying circuit 42) can be displayed to urge the user to take action such as inspection or component replacement.

If, in step S39, the amplification factor Ac is outside the expected range AR, that is, the amplification factor Ac is lower than the lower limit value Acmin of the expected range AR (Ac<Acmin), or the amplification factor Ac is higher than the upper limit value Acmax of the expected range AR (Ac>Acmax) (YES in step S39), the control unit 30 determines that a failure has occurred in the amplifying circuit 42 (step S40), and outputs an amplifying circuit failure signal (step S41). For example, when the amplifying circuit failure signal is inputted to the vehicle-side ECU, a failure indication of the amplifying circuit 42 can be displayed to urge the user to take action such as inspection or component replacement.

### Effects of the Present Embodiment

As described above, the gas sensor 1 according to the present embodiment is a gas sensor 1 for detecting hydrogen gas (to-be-detected gas) contained in a target atmosphere, the gas sensor 1 including: a first detection element 10 and a second detection element 20 connected in series to form one side of a bridge circuit 41, the first detection element 10 being exposed to a reference atmosphere obtained by removing the hydrogen gas from the target atmosphere and having a resistance value changing in response to own temperature change thereof, the second detection element 20 being connected in series to the first detection element 10, exposed to the target atmosphere, and having a resistance value changing in response to own temperature change thereof; an amplifying circuit 42 configured to output an amplified voltage Vd2 on the basis of an intermediate voltage between the first detection element 10 and the second detection element 20 when a predetermined voltage is applied to the bridge circuit 41; a non-amplifying circuit 43 configured to output the intermediate voltage as it is as a non-amplified voltage Vd1; and a control unit 30 to which the amplified voltage Vd2 and the non-amplified voltage Vd1 are inputted, wherein the control unit 30 calculates a hydrogen gas concentration Dc (concentration of the to-be-detected gas) on the basis of the inputted amplified voltage Vd2, and determines a failure of the gas sensor 1 on the basis of at least the non-amplified voltage Vd1.

With the configuration of the present embodiment, the control unit 30 is capable of detecting the concentration of the to-be-detected gas with high sensitivity on the basis of the amplified voltage Vd2. In addition to the presence or absence of a failure in the detection element 10 or 20 or in the amplifying circuit 42, the control unit 30 can determine the location of the failure on the basis of the non-amplified voltage Vd1. In the gas sensor 1 capable of calculating the hydrogen gas concentration D within a predetermined concentration range, by acquiring the non-amplified voltage Vd1 to enable easy monitoring of the intermediate voltage over a wide range, for example, if the calculated hydrogen gas concentration Dc is maintained at the upper limit value Dcmax of the concentration range, the control unit 30 can easily determine whether the calculated hydrogen gas concentration Dc reflects the actual concentration of the to-be-detected gas or is due to a failure of the detection element 10 or 20 or the amplifying circuit 42, on the basis of the non-amplified voltage Vd1. On the basis of the determination result, the control unit 30 can, for example, output a failure signal or a high concentration warning signal to the vehicle-side ECU, and the vehicle-side ECU can display the signal on the display unit 60 to urge the user to take action.

In the gas sensor 1 according to the present embodiment, the non-amplifying circuit 43 is configured to be able to output the non-amplified voltage Vd1 within a predetermined non-amplified output range to the control unit 30, and if the inputted non-amplified voltage Vd1 is a lower limit value Vd1min or an upper limit value Vd1max of the non-amplified output range, the control unit 30 determines that a failure has occurred in at least either the first detection element 10 or the second detection element 20.

With the configuration of the present embodiment, by referring to the non-amplified voltage Vd1, the control unit 30 can determine the presence or absence of a failure of the detection element 10 or 20, which cannot be determined solely from the hydrogen gas concentration Dc calculated from the amplified voltage Vd2. For example, in Process Example 1, if the hydrogen gas concentration Dc which is calculated on the basis of the amplified voltage Vd2 is maintained at the upper limit value Dcmax but the non-amplified voltage Vd1 is neither the lower limit value Vd1min nor the upper limit value Vd1max, the control unit 30 determines that the hydrogen gas concentration D in the target atmosphere is high.

In the gas sensor 1 according to the present embodiment, the control unit 30 calculates an amplification factor Ac on the basis of the inputted amplified voltage Vd2 and the inputted non-amplified voltage Vd1, and determines that a failure has occurred in the amplifying circuit 42, if the amplification factor Ac is outside the expected range AR.

With the configuration of the present embodiment, the control unit 30 can determine the presence or absence of a failure of the amplifying circuit 42 by calculating the amplification factor Ac from the amplified voltage Vd2 and the non-amplified voltage Vd1 and comparing the amplification factor Ac with the expected range AR. For example, in Process Example 2, if the hydrogen gas concentration Dc which is calculated on the basis of the amplified voltage Vd2 is maintained at the upper limit value Dcmax but the amplification factor Ac which is calculated from the amplified voltage Vd2 and the non-amplified voltage Vd1 is within the expected range AR, the control unit 30 determines that the hydrogen gas concentration D in the target atmosphere is high.

### OTHER EMBODIMENTS

(1) In the above embodiment, the case where hydrogen gas is the to-be-detected gas has been described, but the present disclosure is not limited thereto. For example, if the reference atmosphere is sealed in the first housing portion in which the first detection element is placed and only the second detection element is exposed to the target atmosphere, any gas such as methane gas can be the to-be-detected gas.

(2) The material forming each member in the gas sensor of the above embodiment is merely an example, and each member may be formed of another material. The shape of the gas sensor in the above embodiment is also merely an example, and can be changed to various shapes.

(3) Process Example 1 and Process Example 2 described in the above embodiment are merely examples. For example, in the gas sensor 1 according to the above embodiment, the control unit 30 may be configured to first acquire the non-amplified voltage Vd1, confirm that the non-amplified voltage Vd1 is neither the lower limit value Vd1min nor the upper limit value Vd1max (determine that no failure has occurred in the detection elements 10 and 20), then acquire the amplified voltage Vd2, calculate the amplification factor Ac, confirm that the amplification factor Ac is within the expected range AR (determine that no failure has occurred in the amplifying circuit 42), then calculate the hydrogen gas concentration Dc on the basis of the amplified voltage Vd2, and output the hydrogen gas concentration Dc to the display unit 60 to display the hydrogen gas concentration Dc thereon if it is confirmed that the hydrogen gas concentration Dc is not the upper limit value Dcmax (it is determined that the hydrogen gas concentration Dc is not so high that a warning is required).

(4) In Process Example 1 (FIG. 6) described in the above embodiment, if NO is determined in step S14, it is determined that the hydrogen gas concentration Dc is very high, but there is a possibility that the hydrogen gas concentration Dc becomes the upper limit value due to a failure of the amplifying circuit 42 (the concentration of hydrogen gas is not high). Therefore, by executing step S34 and the subsequent steps in Process Example 2 (FIG. 7) instead of step S15 in Process Example 1, it may be determined that the hydrogen gas concentration Dc is very high, if it is determined that no failure has occurred in the amplifying circuit 42. That is, each step in Process Example 1 and Process Example 2 may be combined as appropriate to perform failure determination of the detection elements 10 and 20, failure determination of the amplifying circuit 42, and high concentration determination of hydrogen gas.

### EXPLANATION OF SYMBOLS

1: gas sensor
3: casing
3A: internal space
3B: gas introduction port
3C: filter
3D: inner frame
4: circuit board
5: microcomputer
6: base
7: seal member
10: first detection element
11: first housing portion
11B: first opening
11C: membrane body
12: wire
13A, 13B: first electrode pad
14: temperature measuring resistor
15A, 15B: second electrode pad
16: substrate
17: recess
18: insulating layer
19: first heat generating resistor
20: second detection element
21: second housing portion
21B: second opening
29: second heat generating resistor
30: control unit
39, 49: fixed resistor
41: bridge circuit
42: amplifying circuit
42A: operational amplifier
43: non-amplifying circuit
50: DC power supply
60: display unit
A: amplification factor
AR: expected range (of amplification factor)
Ac: amplification factor (calculated based on Vd1 and Vd2)
Acmax: upper limit value (of amplification factor Ac)
Acmin: lower limit value (of amplification factor Ac)
D: hydrogen gas concentration
Dc: hydrogen gas concentration (calculated based on Vd2)
Dcmax: upper limit value (of hydrogen gas concentration
Dc)
R1, R2: fixed resistor
Vcc: application voltage
Vd0: reference voltage
Vd1: non-amplified voltage (intermediate voltage)
Vd1max: upper limit value (of non-amplified voltage Vd1)
Vd1min: lower limit value (of non-amplified voltage Vd1)
Vd2: non-amplified voltage

1. A gas sensor for detecting a to-be-detected gas contained in a target atmosphere, the gas sensor comprising:
a first detection element and a second detection element connected in series to form one side of a bridge circuit,
the first detection element being exposed to a reference atmosphere obtained by removing the to-be-detected gas from the target atmosphere and having a resistance value changing in response to own temperature change thereof,
the second detection element being connected in series to the first detection element, exposed to the target atmosphere, and having a resistance value changing in response to own temperature change thereof;
an amplifying circuit configured to output an amplified voltage on the basis of an intermediate voltage between the first detection element and the second detection element when a predetermined voltage is applied to the bridge circuit;
a non-amplifying circuit configured to output the intermediate voltage as it is as a non-amplified voltage; and
a control unit to which the amplified voltage and the non-amplified voltage are inputted, wherein
the control unit calculates a concentration of the to-be-detected gas on the basis of the inputted amplified voltage, and determines presence or absence of a failure of the gas sensor on the basis of at least the non-amplified voltage.

2. The gas sensor according to claim 1, wherein
the non-amplifying circuit is configured to be able to output the non-amplified voltage within a predetermined non-amplified output range to the control unit, and
if the inputted non-amplified voltage is a lower limit value or an upper limit value of the non-amplified output range, the control unit determines that a failure has occurred in at least either the first detection element or the second detection element.

3. The gas sensor according to claim 1- or 2, wherein the control unit calculates an amplification factor on the basis of the inputted amplified voltage and the inputted non-amplified voltage, and determines that a failure has occurred in the amplifying circuit, if the amplification factor is outside an expected range.

4. The gas sensor according to claim 2, wherein the control unit calculates an amplification factor on the basis of the inputted amplified voltage and the inputted non-amplified voltage, and determines that a failure has occurred in the amplifying circuit, if the amplification factor is outside an expected range.
